(19) United States
(12) Patent Application Publication
(10) Pub. No.: US 2024/0167075 A1
(43) Pub. Date: May 23, 2024

(54) METHODS FOR ASSAYING ENZYME ACTIVITY

(71) Applicant: Regeneron Pharmaceuticals, Inc., Tarrytown, NY (US)

(72) Inventors: Biao Shen, Dobbs Ferry, NY (US); Ning Li, New Canaan, CT (US)

(21) Appl. No.: 18/389,403

(22) Filed: Nov. 14, 2023

Related U.S. Application Data
(60) Provisional application No. 63/426,206, filed on Nov. 17, 2022.

(51) Int. Cl. C12Q 1/48; B01D 15/38; G01N 30/72; G01N 33/58

(52) U.S. Cl. CPC C12Q 1/48; B01D 15/3814; G01N 30/7233; G01N 33/582; G01N 2030/027

(57) ABSTRACT

The present application describes methods for assaying activity of an enzyme by using a donor substrate, a labeling substrate, and a liquid chromatography column.

### CROSS-REFERENCE TO RELATED APPLICATIONS

This application claims the benefit of U.S. Provisional Application No. 63/426,206, filed Nov. 17, 2022, which is incorporated by reference herein in its entirety.

### FIELD

The present invention generally pertains to methods for assaying enzyme activity. In particular, the present invention generally pertains to methods that use liquid chromatography-mass spectrometry-based quantitation to assay enzyme activity.

### BACKGROUND

Enzymes are large and complex molecules that play a critical role in many biological processes. Abnormal enzyme activity (for example, overexpression, underexpression, or mutation) can cause, contribute to, and/or exacerbate many diseases, such as diabetes, cancers, Alzheimer's disease and human immunodeficiency virus (HIV). Therefore, enzymes also function as biomarkers in clinical medicine.

Enzymes also have been extensively employed in industrial production of products such as sweetening agents, antibiotics, cleaning products, clinical diagnostic assays and environmental applications. Therefore, it is important to detect enzyme activity accurately and sensitively.

Currently available approaches to measure assay enzyme activity monitor the catalysis carried out by the protein using different methods of detection. The commercial kits available to measure enzyme activity are specific to either the enzyme or the donor substrate the enzyme can bind. The present invention provides an efficient method which can be applied to multiple enzymes, multiple detectable labels, and multiple donor substrates.

### SUMMARY

Embodiments disclosed herein satisfy the aforementioned demands by providing methods for assaying activity of an enzyme.

In one exemplary embodiment, the method can comprise contacting said enzyme with a donor substrate and a labeling substrate to form a sample; incubating said sample for a predetermined amount of time; contacting said incubated sample to a liquid chromatography column; quantifying an amount of said labeling substrate obtained from said liquid chromatography column to assay the activity of the enzyme.

In one aspect, the method can further comprise contacting a mixture of said donor substrate and said labeling to a substrate liquid chromatography column and quantifying the amount of said labeling substrate in said incubated sample. In another aspect, the sample can be incubated for two or more predetermined amounts of time and the two or more incubated samples are evaluated by contacting said incubated sample to a liquid chromatography column and quantifying amount of said labeling substrate in said incubated sample after contacting said chromatography column.

In one aspect, the enzyme can be a mutated version of a naturally occurring enzyme. In another aspect, the enzyme can be a naturally occurring enzyme. In another aspect, the enzyme can be non-naturally occurring. In a specific aspect, the enzyme can be Beta-1,4-galactosyltransferase 1 (β4GALT1).

In one aspect, the enzyme can catalyze glycosylation. In another aspect, the enzyme can catalyze transfer of a sugar molecule onto a protein. In yet another aspect, the enzyme can catalyze transfer of a functional group onto a protein. Non-limiting examples of functional groups can include single carbon groups, aldehyde groups, ketone groups, acyl groups, glycosyl groups, alkyl groups, aryl groups, nitrogenous groups, phosphorous-containing groups, sulfur-containing groups, selenium-containing groups, and/or heavy metal-containing groups.

In one aspect, the enzyme can catalyze glycosylation, the donor substrate can provide a sugar molecule and said labeling substrate can be capable of binding to the sugar molecule of the donor substrate.

In one aspect, said labeling substrate can be detected by use of mass spectrometry, fluorescence, chemiluminescence, bioluminescence, electrochemiluminescence, and/or radioactivity. In a specific aspect, said labeling substrate can include 2-aminobenzamide.

In one aspect, said labeling substrate can be quantified. Non-limiting examples of such methods include use of mass spectrometry, UV-vis absorbance spectroscopy, fluorescence spectroscopy, scintillation counter, autoradiography, and electron microscopy.

In one aspect, said labeling substrate can be quantified using mass spectrometry. In one aspect, said mass spectrometry can be coupled online with said liquid chromatography column. In a specific aspect, said liquid chromatography column coupled with mass spectrometry can be run under native conditions.

In one aspect, said liquid chromatography column can be an anion exchange column, a cation exchange column, a hydrophobic interaction chromatography column, a hydrophilic interaction chromatography column, a mixed mode chromatography column, or a protein A chromatography column.

In one aspect, the predetermined amount of time can be selected from about 0 minutes, about 2 minutes, about 5 minutes, about 10 minutes, about 15 minutes, about 20 minutes, about 25 minutes, about 30 minutes, about 45 minutes, or about 60 minutes.

In one aspect, said quantification can be performed for three unique predetermined amounts of time to assay the activity of the enzyme.

In one exemplary embodiment, the method can comprise contacting said enzyme with a donor substrate and a labeling substrate to form a sample, wherein said donor substrate and said labeling substrate can form a product including at least a part of the labeling substrate and at least a part of the donor substrate; incubating said sample for a predetermined amount of time; contacting said incubated sample to a liquid chromatography column; quantifying an amount of said product to assay the activity of the enzyme.

In one aspect, the method can further comprise contacting a mixture of said donor substrate and said labeling substrate to a liquid chromatography column and quantifying the amount of said product in said incubated sample. In another aspect, the sample can be incubated for two or more predetermined amounts of time and the two or more incubated samples are evaluated by contacting said incubated samples to a liquid chromatography column and quantifying the amount of said product in said incubated samples after contacting said chromatography column.

In one aspect, the enzyme can be a mutated version of a naturally occurring enzyme. In another aspect, the enzyme can be a naturally occurring enzyme. In another aspect, the enzyme can be a non-naturally occurring enzyme. In a specific aspect, the enzyme can be β4GALT1.

In one aspect, the enzyme can catalyze glycosylation. In another aspect, the enzyme can catalyze transfer of a sugar molecule onto a protein. In yet another aspect, the enzyme can catalyze transfer of a functional group onto a protein. Non-limiting examples of functional groups can include single carbon groups, aldehyde groups, ketone groups, acyl groups, glycosyl groups, alkyl groups, aryl groups, nitrogenous groups, phosphorous-containing groups, sulfur-containing groups, selenium-containing groups, and/or heavy metal-containing groups.

In one aspect, the enzyme can catalyze glycosylation, the donor substrate can provide a sugar molecule and the labeling substrate can be capable of binding to the sugar molecule of the donor substrate.

In one aspect, said labeling substrate can be capable of being detected by use of mass spectrometry, fluorescence, chemiluminescence, bioluminescence, electrochemiluminescence, and/or radioactivity. In a specific aspect, said labeling substrate can include 2-aminobenzamide.

In one aspect, said labeling substrate can be quantified. Non-limiting examples of such methods include use of mass spectrometry, UV-vis absorbance spectroscopy, fluorescence spectroscopy, scintillation counter, autoradiography, and electron microscopy.

In one aspect, said product can be quantified using mass spectrometry. In one aspect, said mass spectrometry can be coupled online with said liquid chromatography column. In a specific aspect, said liquid chromatography column coupled with mass spectrometry can be run under native conditions.

In one aspect, said liquid chromatography column can be an anion exchange column, a cation exchange column, a hydrophobic interaction chromatography column, a hydrophilic interaction chromatography column, a mixed mode chromatography column, or a protein A chromatography column.

In one aspect, the predetermined amount of time can be selected from about 0 minutes, about 2 minutes, about 5 minutes, about 10 minutes, about 15 minutes, about 20 minutes, about 25 minutes, about 30 minutes, about 45 minutes, or about 60 minutes.

In one aspect, said quantification can be performed for three unique predetermined amounts of time to assay the activity of the enzyme.

This disclosure also provides methods for quantifying or characterizing the activity of a glycosyltransferase. In some exemplary embodiments, the methods can comprise (a) incubating a mixture including a glycosyl donor, a glycosyl acceptor, and a glycosyltransferase, wherein said glycosyl acceptor includes a fluorescent label, and wherein said glycosyltransferase is capable of transferring a saccharide from said glycosyl donor to said glycosyl acceptor to form a product; (b) subjecting said incubated mixture to chromatographic separation to separate said glycosyl acceptor from said product; and (c) subjecting said separated glycosyl acceptor and said separated product to fluorescence spectroscopy to quantify or characterize the activity of said glycosyltransferase.

In one aspect, the glycosyl donor is uridine diphosphate galactose. In another aspect, the glycosyl acceptor is a glycan. In an additional aspect, the glycosyl acceptor is a G0F glycan.

In one aspect, the glycosyltransferase is Beta-1,4-galactosyltransferase 1. In another aspect, the fluorescent label is 2-aminobenzamide. In an additional aspect, the saccharide is galactose.

In one aspect, the chromatographic separation is hydrophilic interaction chromatography separation.

In one aspect, the method further comprises subjecting the incubated mixture to ultraviolet detection. In another aspect, the method further comprises subjecting said separated glycosyl acceptor and/or said separated product to mass spectrometry analysis.

In one aspect, a duration of said incubating is from about 0 minutes to about 60 minutes, about 0 minutes, about 1 minute, about 2 minutes, about 5 minutes, about 10 minutes, about 15 minutes, about 25 minutes, about 30 minutes, about 40 minutes, about 50 minutes, or about 60 minutes.

These, and other, aspects of the invention will be better appreciated and understood when considered in conjunction with the following description and accompanying drawings. The following description, while indicating various aspects and numerous specific details thereof, is given by way of illustration and not of limitation. Many substitutions, modifications, additions, or rearrangements may be made within the scope of the invention.

### BRIEF DESCRIPTION OF THE DRAWINGS

FIG. 1 shows a schematic of an exemplary embodiment of the present invention.

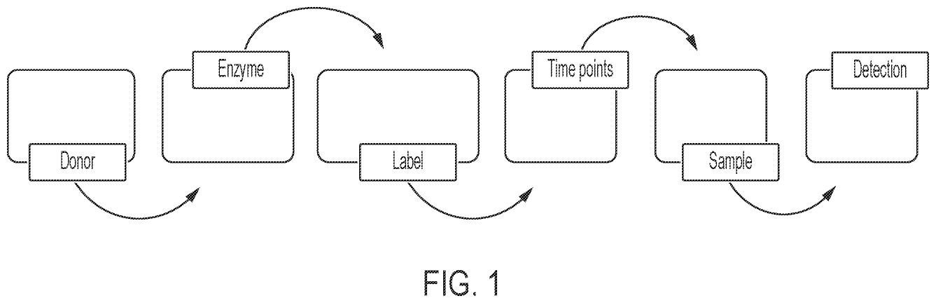

FIG. 2 shows the function of glycosyltransferase enzymes in the Golgi apparatus of the cell, according to an exemplary embodiment.

FIG. 3 illustrates a workflow for assaying β4GALT1 enzyme activity by using a donor substrate and acceptor substrate, according to an exemplary embodiment.

FIG. 4 shows the specific activity for β4GALT1 wildtype and β4GALT1 with N352S mutation, determined using a commercial kit, according to an exemplary embodiment.

FIG. 5A shows the UV absorbance peaks of UDP-Galactose detected at 280 nm after incubation with wildtype or mutant β4GALT1, according to an exemplary embodiment.

FIG. 5B shows the UV absorbance peak area of UDP-Galactose after incubation with wildtype or mutant β4GALT1, according to an exemplary embodiment.

FIG. 6 shows the peak area from an extracted chromatogram of GlcNAc-Galactose after incubation with wildtype or mutant β4GALT1, according to an exemplary embodiment.

FIG. 7 shows a schematic of the reaction principle for an exemplary embodiment of the present invention.

FIG. 8 shows fluorescence (FLR) signal for 2-AB labeled G0F and 2-AB labeled G1F and G2F (product) after incubation with wildtype β4GALT1, according to an exemplary embodiment.

FIG. 9 shows mass spectra for 2-AB labeled G0F, G1F and G2F after incubation with wildtype β4GALT1, according to an exemplary embodiment.

FIG. 10 shows the change in signal intensity versus time for samples incubated over time due to sample evaporation, according to an exemplary embodiment.

FIG. 11 shows the FLR signal for 2-AB-G0F at 0 minutes, 20 minutes, 40 minutes, and 60 minutes using experimental conditions but without the addition of β4GALT1, according to an exemplary embodiment.

FIG. 12 shows the FLR signal for 2-AB labeled G0F, G1F, and G2F after incubation with wildtype or mutant β4GALT1, according to an exemplary embodiment.

FIG. 13 shows the FLR peak area of 2-AB labeled G0F, G1F, and G2F after incubation for varying periods of time with wildtype or mutant β4GALT1, according to an exemplary embodiment.

### DETAILED DESCRIPTION

Enzymes are the most diverse and largest group of proteins. Enzymes play an important role in regulation of metabolic steps within a cell.

Enzymatic assays can be based on measuring the rate of the reaction catalyzed by an enzyme. The reaction rate can be determined based on the consumption of the substrate or the generation of a product over a given time. The progress of the reaction can be monitored continuously (continuous assay) using spectroscopic or electrochemical techniques revealing full progress of the reaction. An alternative to continuous monitoring is stopping the reaction and measuring the total amount of product formed or substrate consumed within the given reaction time by a subsequent chemical indicator reaction or a separation method (stopped assay). Both the continuous and stopped assay use an acceptor substrate (a protein) for the enzyme, wherein the enzyme can catalyze the transformation of the acceptor substrate. For such assays, the substrate concentrations need to be sufficiently high to saturate the enzyme, which ensures operation at its maximum rate.

A commercial kit can also be used to assay enzyme activity. However, commercially available kits are generally specific to a particular type of enzyme. For example, a Glycotransferase Activity Kit is used for assaying the enzyme activity of glycosyltransferases using diphosphonucleotide sugars as donor substrates (glycosyl donors). Therefore, the Glycotransferase Activity Kit cannot be modified or used for enzymes that do not utilize diphosphonucleotide sugars as donor substrates. The Glycotransferase Activity Kit also needs an additional specific phosphatase enzyme for the assay to properly function. The present invention provides methods that can be continuous in nature and can be applied to different enzymes and/or use different labels.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described. All publications mentioned are hereby incorporated by reference.

The term “a” should be understood to mean “at least one”; and the terms “about” and “approximately” should be understood to permit standard variation as would be understood by those of ordinary skill in the art; and where ranges are provided, endpoints are included.

As used herein, the term “chromatography” refers to a process in which a chemical mixture carried by a liquid or gas can be separated into components as a result of differential distribution of the chemical entities as they flow around or over a stationary liquid or solid phase. Non-limiting examples of chromatography include traditional reversed-phased (RP), ion exchange (IEX) and normal phase chromatography (NP). Unlike RP, NP and IEX chromatography, in which hydrophobic interaction, hydrophilic interaction and ionic interaction respectively are the dominant interaction modes, mixed-mode chromatography can employ a combination of two or more of these interaction modes. Several types of liquid chromatography can be used with the mass spectrometer, such as, for example, rapid resolution liquid chromatography (RRLC), ultra-performance liquid chromatography (UPLC), ultra-fast liquid chromatography (UFLC) and nano liquid chromatography (nLC). For further details on chromatography method and principles, see Colin et al. (COLIN F. POOLE ET AL., LIQUID CHROMATOGRAPHY FUNDAMENTALS AND INSTRUMENTATION (2017)). Non-limiting examples of chromatography include gas chromatography, reverse phase liquid chromatography, ion-exchange chromatography, size exclusion chromatography, affinity chromatography, hydrophobic interaction chromatography, hydrophilic interaction chromatography, or mixed-mode chromatography.

As used herein, “affinity chromatography” can include separations including any method by which two substances are separated based on their affinity to chromatographic material. It can comprise subjecting the substances to a column comprising a suitable affinity chromatographic media. Non-limiting examples of such chromatographic media include, but are not limited to, Protein A resin, Protein G resin, affinity supports comprising the antigen against which the binding molecule was raised, and affinity supports comprising an Fc binding protein. In one aspect, an affinity column can be equilibrated with a suitable buffer prior to sample loading. An example of a suitable buffer can be a Tris/NaCl buffer, pH around 7.2. Following this equilibration, the sample can be loaded onto the column. Following the loading of the column, the column can be washed one or multiple times using, e.g., the equilibrating buffer. Other washes including washes employing different buffers can be used before eluting the column. The affinity column can then be eluted using an appropriate elution buffer. An example of a suitable elution buffer can be an acetic acid/NaCl buffer, pH around 3.5. The eluate can be monitored using techniques well known to those skilled in the art. For example, the absorbance at 280 nm wavelength can be followed.

As used herein, “ion exchange chromatography” can include separations including any method by which two substances are separated based on the difference in their respective ionic charges, either on the molecule of interest and/or chromatographic material as a whole, or locally on specific regions of the molecule of interest and/or chromatographic material, and thus can employ either cationic exchange material or anionic exchange material. Ion exchange chromatography separates molecules based on differences between the local charges of the molecules of interest and the local charges of the chromatographic material. A packed ion-exchange chromatography column or an ion-exchange membrane device can be operated in a bind-elute mode, a flow-through mode, or a hybrid mode. After washing the column or the membrane device with the equilibration buffer or another buffer with different pH and/or conductivity, the product recovery can be achieved by increasing the ionic strength (i.e., conductivity) of the elution buffer to compete with the solute for the charged sites of the ion exchange matrix. Changing the pH and thereby altering the charge of the solute can be another way to achieve elution of the solute. The change in conductivity or pH may be gradual (gradient elution) or stepwise (step elution). The column can then be regenerated before the next use. Anionic or cationic substituents may be attached to matrices in order to form anionic or cationic supports for chromatography. Non-limiting examples of anionic exchange substituents include diethylaminoethyl (DEAE), quaternary aminoethyl (QAE) and quaternary amine (Q) groups. Cationic substituents include carboxymethyl (CM), sulfoethyl (SE), sulfopropyl (SP), phosphate (P) and sulfonate (S). Cellulose ion exchange medias or support can include DE23™, DE32™, DE52™, CM-23™, CM-32™, and CM-52™, which are available from Whatman Ltd. Maidstone, Kent, U.K. SEPHADEX®-based and -locross-linked ion exchangers are also known. For example, DEAE-, QAE-, CM-, and SP-SEPHADEX® and DEAE-, Q-, CM- and 5-SEPHAROSE® and SEPHAROSE® Fast Flow, and Capto™ S are all available from GE Healthcare. Further, both DEAE and CM derivatized ethylene glycol-methacrylate copolymer such as TOYOPEARL™ DEAE-650S or M and TOYOPEARL™ CM-650S or M are available from Toso Haas Co., Philadelphia, Pa., or Nuvia S and UNOSphere™ S from BioRad, Hercules, Calif., Eshmuno® S from EMD Millipore, MA.

As used herein, the term “hydrophobic interaction chromatography resin” can include a solid phase which can be covalently modified with phenyl, octyl, or butyl chemicals. It can use the properties of hydrophobicity to separate molecules from one another. In this type of chromatography, hydrophobic groups such as, phenyl, octyl, hexyl or butyl can be attached to the stationary column. Molecules that pass through the column that have hydrophobic amino acid side chains on their surfaces are able to interact with and bind to the hydrophobic groups on the column. Examples of hydrophobic interaction chromatography resins or support include Phenyl sepharose FF, Capto Phenyl (GE Healthcare, Uppsala, Sweden), Phenyl 650-M (Tosoh Bioscience, Tokyo, Japan) and Sartobind Phenyl (Sartorius corporation, New York, USA).

As used herein, the term “Mixed Mode Chromatography (MMC)” or “multimodal chromatography” includes a chromatographic method in which solutes interact with stationary phase through more than one interaction mode or mechanism. MMC can be used as an alternative or complementary tool to traditional reversed-phased (RP), ion exchange (IEX) and normal phase chromatography (NP). Unlike RP, NP and IEX chromatography, in which hydrophobic interaction, hydrophilic interaction and ionic interaction respectively are the dominant interaction modes, mixed-mode chromatography can employ a combination of two or more of these interaction modes. Mixed mode chromatography media can provide unique selectivity that cannot be reproduced by single mode chromatography. Mixed mode chromatography can also provide potential cost savings, longer column lifetimes and operation flexibility compared to affinity-based methods. In some exemplary embodiments, the mixed mode chromatography media can be comprised of mixed mode ligands coupled to an organic or inorganic support, sometimes denoted a base matrix, directly or via a spacer. The support may be in the form of particles, such as essentially spherical particles, a monolith, filter, membrane, surface, capillaries, etc. In some specific exemplary embodiments, the support can be prepared from a native polymer, such as cross-linked carbohydrate material, such as agarose, agar, cellulose, dextran, chitosan, konjac, carrageenan, gellan, alginate and the like. To obtain high adsorption capacities, the support can be porous, and ligands are then coupled to the external surfaces as well as to the pore surfaces. Such native polymer supports can be prepared according to standard methods, such as inverse suspension gelation (S Hjerten: Biochim Biophys Acta 79(2), 393-398 (1964). Alternatively, the support can be prepared from a synthetic polymer, such as cross-linked synthetic polymers, e.g., styrene or styrene derivatives, divinylbenzene, acrylamides, acrylate esters, methacrylate esters, vinyl esters, vinyl amides and the like. Such synthetic polymers can be produced according to standard methods, see e.g., “Styrene based polymer supports developed by suspension polymerization” (R Arshady: Chimica e L'Industria 70(9), 70-75 (1988)). Porous native or synthetic polymer supports are also available from commercial sources, such as Amersham Biosciences, Uppsala, Sweden.

The term “hydrophilic interaction chromatography” or HILIC is intended to include a process employing a hydrophilic stationary phase and a hydrophobic organic mobile phase in which hydrophilic compounds are retained longer than hydrophobic compounds. In certain embodiments, the process utilizes a water-miscible solvent mobile phase. In certain embodiments, the term also includes ERLIC (electrostatic repulsion hydrophilic interaction chromatography), Cationic ERLIC and Anionic ERLIC.

In some exemplary embodiments, the chromatography is coupled on-line with a mass spectrometer. As used herein, the term “mass spectrometer” includes a device capable of identifying specific molecular species and measuring their accurate masses. The term is meant to include any molecular detector into which a polypeptide or peptide may be eluted for detection and/or characterization. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends heavily on the application.

In some exemplary embodiments, the mass spectrometer can be a tandem mass spectrometer.

As used herein, the term “tandem mass spectrometry” includes a technique where structural information on sample molecules is obtained by using multiple stages of mass selection and mass separation. A prerequisite is that the sample molecules can be transferred into gas phase and ionized intact and that they can be induced to fall apart in some predictable and controllable fashion after the first mass selection step. Multistage MS/MS, or MSⁿ, can be performed by first selecting and isolating a precursor ion (MS²), fragmenting it, isolating a primary fragment ion (MS³), fragmenting it, isolating a secondary fragment (MS⁴), and so on as long as one can obtain meaningful information, or the fragment ion signal is detectable. Tandem MS has been successfully performed with a wide variety of analyzer combinations. What analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device.

In some exemplary embodiments, the mass spectrometer can be coupled to a nano liquid chromatography. In some exemplary embodiments, the mobile phase used to elute the protein in liquid chromatography can be a mobile phase that can be compatible with a mass spectrometer. In some specific exemplary embodiments, the mobile phase can be ammonium acetate, ammonium bicarbonate, or ammonium formate, or combinations thereof.

In some exemplary embodiments, the mass spectrometer can be coupled to a liquid chromatography-multiple reaction monitoring system.

As used herein, “multiple reaction monitoring” or “MRM” refers to a mass spectrometry-based technique that can precisely quantify small molecules, peptides, and proteins within complex matrices with high sensitivity, specificity and a wide dynamic range (Paola Picotti & Ruedi Aebersold, Selected reaction monitoring-based proteomics: workflows, potential, pitfalls and future directions, 9 NATURE METHODS 555-566 (2012)). MRM can be typically performed with triple quadrupole mass spectrometers wherein a precursor ion corresponding to the selected small molecules/peptides is selected in the first quadrupole and a fragment ion of the precursor ion was selected for monitoring in the third quadrupole (Yong Seok Choi et al., Targeted human cerebrospinal fluid proteomics for the validation of multiple Alzheimers disease biomarker candidates, 930 JOURNAL OF CHROMATOGRAPHY B 129-135 (2013)).

In some exemplary embodiments, the mass spectrometer can be coupled to a liquid chromatography-selected reaction monitoring system.

In some embodiments, the enzyme is capable of catalyzing transformation of a protein. As used herein, the term “protein” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. “Synthetic peptides or polypeptides” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may contain one or multiple polypeptides to form a single functioning biomolecule. A protein can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies.

In another exemplary aspect, a protein can include antibody fragments, nanobodies, recombinant antibody chimeras, cytokines, chemokines, peptide hormones, and the like. Proteins may be produced using recombinant cell-based production systems, such as the insect bacculovirus system, yeast systems (e.g., Pichia sp.), mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation,” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012)). In some embodiments, proteins comprise modifications, adducts, and other covalently linked moieties. These modifications, adducts and moieties include for example avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as, globular proteins and fibrous proteins; conjugated proteins, such as, nucleoproteins, glycoproteins, mucoproteins, chromoproteins, phosphoproteins, metalloproteins, and lipoproteins; and derived proteins, such as, primary derived proteins and secondary derived proteins.

As used herein, “label” or “detectable label” or “labeling substrate” are used interchangeably. It refers to a moiety attached to a specific binding member or analyte to render the reaction between the specific binding member and the analyte detectable, and the specific binding member or analyte so labeled is referred to as “detectably labeled.” A label can produce a signal that is detectable by visual or instrumental means. Various labels include: (i) a tag attached to a specific binding member or analyte by a cleavable linker; or (ii) signal-producing substance, such as chromagens, fluorescent compounds, enzymes, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling.

In certain embodiments, the detectable label includes a chromagen, a fluorescent compound, an enzyme, a chemiluminescent compound or a radioactive compound.

Any detectable label can be used in the method of the invention. An exemplary detectable label is a fluorescent label. An exemplary fluorescent label is an aromatic amine, e.g., 2-aminobenzoic acid, 2-aminobenzamide, 2-aminopyridine, 8-aminonapthaline-1,3,6-trisulfonic acid, 2-aminoacridone, and 9-aminopyrene-1,3,6-trisulfonic acid. Further, any suitable signal-producing substance known in the art can be used as a detectable label. For example, the detectable label can be a radioactive label (such as 3H, 14C, 32P, 33P, 35S, 90Y, 99Tc, 111In, 125I, 131I, 177Lu, 166Ho, and 153Sm), an enzymatic label (such as horseradish peroxidase, alkaline peroxidase, glucose 6-phosphate dehydrogenase, and the like (if enzymes are used then a corresponding enzymatic substrate must also be added)), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2nd ed., Springer Verlag, N.Y. (1997), and in Haugland, Handbook of Fluorescent Probes and Research Chemicals (1996), which is a combined handbook and catalogue published by Molecular Probes, Inc., Eugene, Oreg. A fluorescent label can be used in FPIA (see, e.g., U.S. Pat. Nos. 5,593,896, 5,573,904, 5,496,925, 5,359,093, and 5,352,803, which are hereby incorporated by reference in their entireties). An acridinium compound can be used as a detectable label in a homogeneous chemiluminescent assay (see, e.g., Adamczyk et al., Bioorg. Med. Chem. Lett. 16: 1324-1328 (2006); Adamczyk et al., Bioorg. Med. Chem. Lett. 4: 2313-2317 (2004); Adamczyk et al., Biorg. Med. Chem. Lett. 14: 3917-3921 (2004); and Adamczyk et al., Org. Lett. 5: 3779-3782 (2003)).

In some exemplary embodiments, said sample is incubated for a predetermined amount of time. For example, the sample can be incubated for about 0 minutes, about 1 minutes, about 2 minutes, about 5 minutes, about 10 minutes, about 15 minutes, about 20 minutes, about 25 minutes, about 30 minutes, about 45 minutes, about 60 minutes, about 75 minutes, about 90 minutes, or about 120 minutes. The time can be chosen depending on the rate of enzyme activity. In another aspect, the predetermined amount of time can be selected based on time intervals. For example, the predetermined amount of time can be about 0 minutes, about 5 minutes, about 10 minutes, about 15 minutes, about 20 minutes, and so on.

As used herein, the term “glycosyltransferase” refers to an enzyme that catalyzes the transfer of a saccharide (which can also be referred to as a sugar or a carbohydrate) from a donor substrate (or glycosyl donor) to an acceptor substrate (or glycosyl acceptor). Examples of glycosyl donors include, for example, sugar nucleotide donors such as uridine diphosphate glucose (UDP-glucose), UDP-galactose, UDP-GlcNAc, UDP-GalNAc, UDP-xylose, UDP-glucuronic acid, guanosine diphosphate (GDP)-mannose, GDP-fucose, or cytidine monophosphate (CMP)-sialic acid, and non-nucleotide donors such as dolichol or polyprenol pyrophosphate.

It is understood that the present invention is not limited to any of the above mentioned enzyme(s), chromatographic resin(s), labeling substrate(s), instrument(s), donor substrate(s), or mass spectrometry system(s) used for identification, and any enzyme(s), chromatographic resin(s), labeling substrate(s), instrument(s), donor substrate(s), mass spectrometry system(s) used for the present invention can be selected by any suitable means.

Various publications, including patents, patent applications, published patent applications, accession numbers, technical articles and scholarly articles are cited throughout the specification. Each of these cited references is incorporated by reference, in its entirety and for all purposes, herein.

The present invention will be more fully understood by reference to the following Examples. They should not, however, be construed as limiting the scope of the invention.

### EXAMPLES

### Example 1. In Vitro Assay to Measure the Activity of β4GALT1 Enzyme

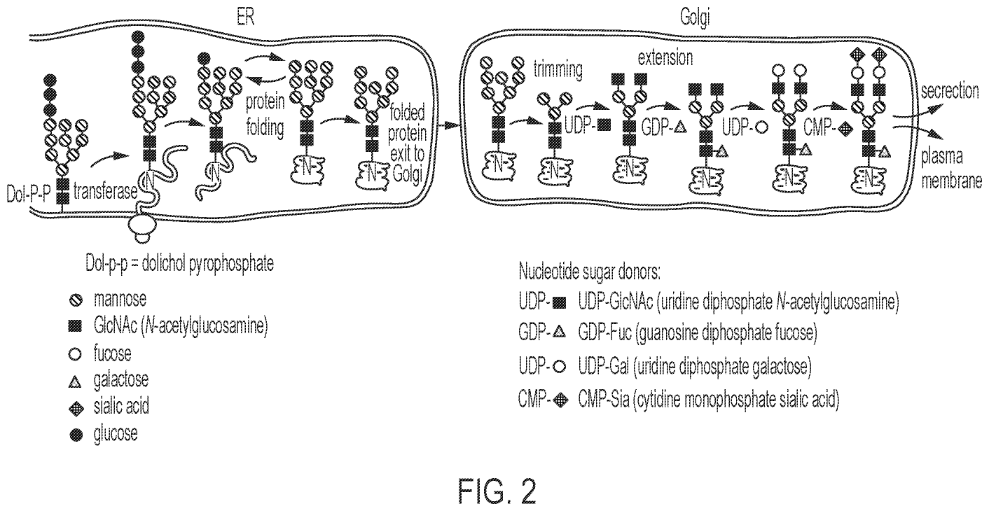

β4GALT1 belongs to the glycosyltransferases super family of enzymes, many of which reside in the Golgi apparatus of the cell. FIG. 2 shows the role of β4GALT1 in transferring galactose to an acceptor sugar molecule. β4GALT1 contributes to the synthesis of N-linked (and O-linked) glycan chains, transferring galactose from UDP-galactose to N-acetylglucosamine (GlcNAc) in the presence of Mn⁺. Thus, impairment of β4GALT1 activity has the potential to alter the structure of N-linked oligosaccharides and introduce aberrations in the glycan structure that may alter glycoprotein function.

A recently published study identified a missense variant (rs551564683, p.Asn352Ser) in β4GALT1. See Montasser et al. Genetic and Genetic and functional evidence links a missense variant in B4GALT1 to lower LDL and fibrinogen. SCIENCE 374, 374 (2021). The enzyme activity of the wild type and the missense variant was assessed.

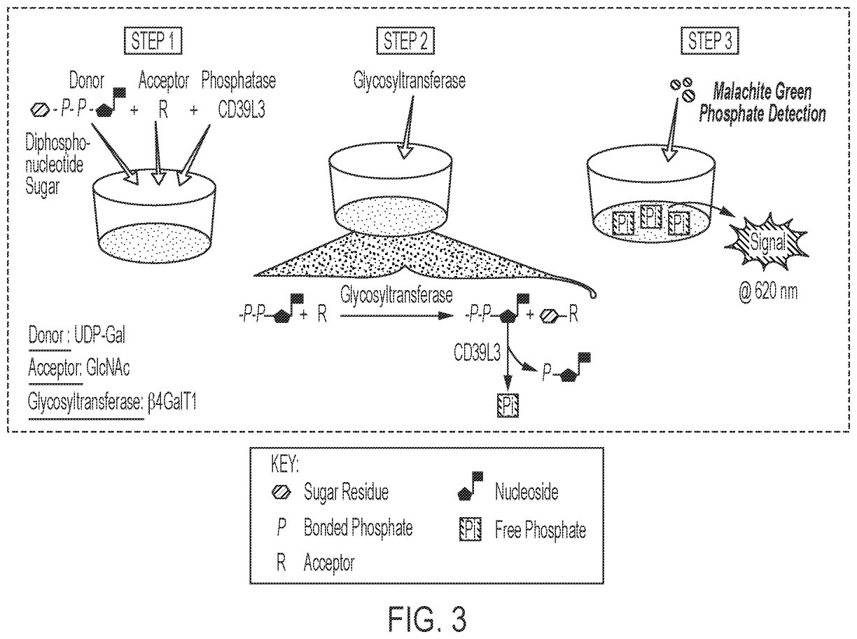

The commercial Phosphate Glycosyltransferase Activity kit was purchased from R&D Systems. The principle of the assay along with the materials provided were utilized according to manufacturer's conditions. In brief, a diphosphonucleotide sugar (such as UDP-Gal) is used as a glycosyl donor and mixed with a glycosyl acceptor, a phosphatase, and a glycosyltransferase. Glycosyltransferase activity exposes a phosphate of the glycosyl donor to phosphatase activity, releasing free phosphate. The amount of phosphate present should be directly proportional to the amount of formed glycosylated product. The kit also uses a specific phosphatase to remove an inorganic phosphate quantitatively from UDP. The released inorganic phosphate is subsequently quantitated by sensitive colorimetric malachite green phosphate detecting reagents. The amount of inorganic phosphate released by the coupling phosphatase should be equal to the nucleotide sugar consumed or glycoconjugate product generated. Therefore, the rate of inorganic phosphate products should reflect the kinetics of a glycosyltransferase reaction (specific activity of the enzyme). The steps as performed are outlined in FIG. 3.

To estimate the amount of UDP produced in the enzymatic reaction, a phosphate standard curve was established according to manufacturer's conditions. The preparations and dilution were also performed as noted in the manual for the kit.

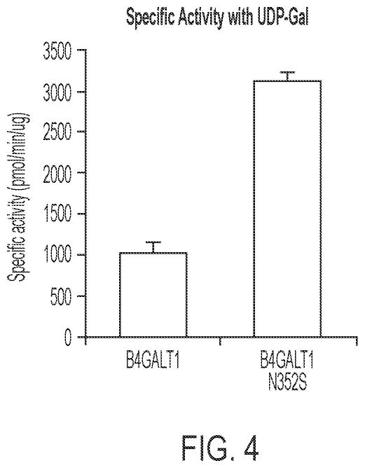

The assay showed that the wildtype β4GALT1 was less active than β4GALT1 with N352S mutation. See FIG. 4. These results were contrary to the results published in previous literature, for example in Montasser et al. (supra), which noted that that the enzymatic activity of β4GALT1 with N352S mutation was less than the wildtype enzyme. Therefore, improved approaches to quantifying β4GALT1 activity were explored.

### Example 2. HILIC-UV/MS Based Assay to Measure the Activity of β4GALT1 Enzyme

Montasser et al. attempted to assay enzyme activity using a method involving quantification of the β4GALT1 substrate UDP-Gal, by measuring its peak area using hydrophilic interaction chromatography-ultraviolet detection-mass spectroscopy. The method as outlined in Montasser et al. was followed, using HILIC-UV-MS to quantitate the decrease of UDP-Gal as a measure of β4GALT1 activity. In particular, the decrease of UDP-Gal could be measured using UV detection and the corresponding increase of GlcNAc-Gal could be measured using MS.

The glycosyl donor was 1.7 nmol UDP-Gal; the glycosyltransferase was 1.1 pmol β4GALT1; and the glycosyl acceptor was 500 nmol GlcNAc. All samples were analyzed using an Acquity UPLC I-Class coupled with Vion IMS Q-Tof (Waters, Milford, MA, USA). A Waters Acquity UPLC Glycoprotein BEH Amide chromatographic column (300 Å, 150 mm×2.1 mm, 1.7 μm) was used to separate the analytes with a column temperature of 45° C. and a flow rate of 0.2 mL/minute. Mobile phase A was 50 mM ammonium acetate at a pH 7.5, and mobile phase B was 5% (v/v) 25 mM ammonium acetate at a pH 7.5 in acetonitrile. A 1 μL aliquot of each mixture (wildtype and mutant β4GALT1) was injected for analysis.

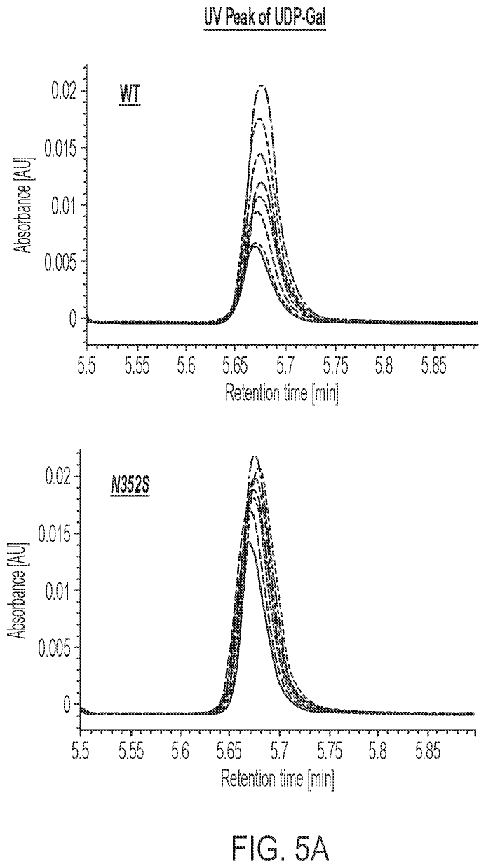

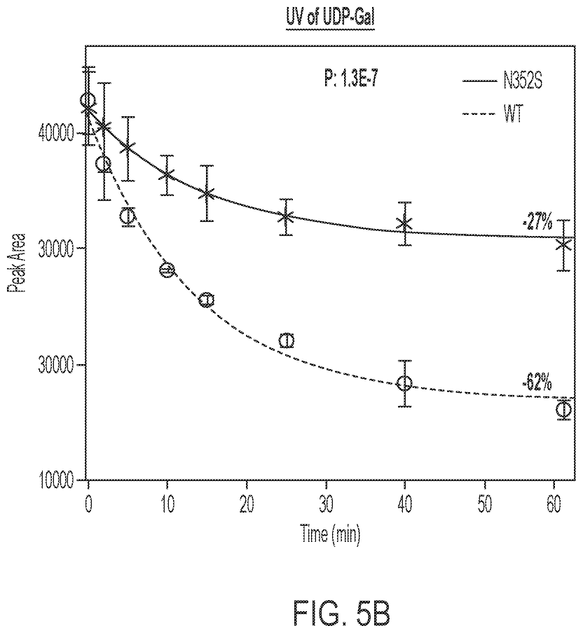

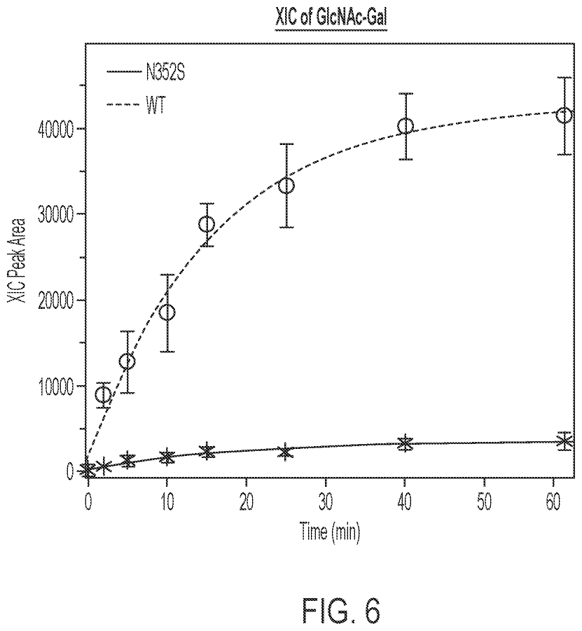

The difference in activity between the wildtype and mutant β4GALT1 was tested using paired two-sided Student's t-test. The results are shown in FIG. 5 and FIG. 6.

FIG. 5 shows the peak area of UDP-Gal detected on a UV detector in samples at different time points (2, 5, 10, 15, 25, 40, and 60 minutes). The results show that the decrease in UDP-Gal is significantly more for the wildtype β4GALT1 compared to the mutant β4GALT1, suggesting that the wildtype β4GALT1 has higher activity. FIG. 6 shows the concentration of the product of the reaction catalyzed by β4GALT1 (GlcNAc-Gal) detected in the samples at different time points (2, 5, 10, 15, 25, 40, and 60 minutes) using MS. The results show that the increase in the amounts of the product over time is significantly more for the wildtype β4GALT1 compared to the mutant β4GALT1. These results are similar to the findings published in Montasser et al.

However, the method of Montasser et al. is limited because UV detection cannot detect glycans themselves, only the change in the substrate, UDP-Gal. It would be preferable to detect both the substrate and the product with a single measurement, and it would further be preferable for a method to be generalizable across any glycosyltransferase. Therefore, new methods for glycosyltransferase characterization were developed.

### Example 3. LC-MS Based Assay to Measure the Activity of β4GALT1 Enzyme

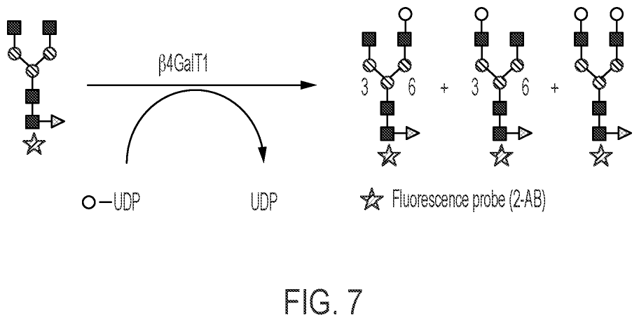

A new method for characterizing glycan transfer activity was developed, using a fluorescent probe, 2-aminobenzamide (2-AB), to label glycans, as shown in FIG. 7, and fluorescence detection (FLR) in combination with HILIC-MS to detect the labeled glycans. Specifically, the enzymatic activities were measured using uridine diphosphate galactose (UDP-Gal) as a donor substrate and a labeled glycan (2-AB-G0F) as the labeling substrate. The experiment was carried out over eight different time points.

The enzymatic reaction was initiated by adding 1.1 pmol of B4GALT1 into a mixture of 1.7 nmol UDP-Gal (the glycosyl donor) and 10 pmol of 2-AB-G0F (the glycosyl acceptor), and the mixture was incubated at room temperature for 2, 5, 10, 15, 25, 40, and 60 minutes. The sample representing the initial time point (0 min) was prepared by omitting the enzyme. This HILIC-FLR-MS based method was used to quantitate both the decrease of UDP-Gal and the increase in formation of the products (G1F/G2F).

All samples were analyzed using an Acquity UPLC I-Class coupled with Vion IMS Q-Tof (Waters, Milford, MA, USA). A Waters Acquity UPLC Glycoprotein BEH Amide chromatographic column (300 Å, 150 mm×2.1 mm, 1.7 μm) was used to separate the analytes with a column temperature of 60° C. and a flow rate of 0.5 mL/minute. Mobile phase A was 50 mM ammonium acetate at a pH 4.4, and mobile phase B was acetonitrile. A 5 μL aliquot of each mixture was injected onto the column for HILIC-MS analysis. The sample run time was 12 minutes.

The samples were monitored using a UV detector at a wavelength of 280 nm and fluorescence with excitation at a wavelength of 330 nm and emission at a wavelength of 420 nm. The Waters Vion IMS Q-Tof mass spectrometer was operated in positive mode and the mass spectra were acquired with a m/z range between 600 and 2000. UDP-Gal was quantitated and the data were collected and processed using UNIFI software (Waters).

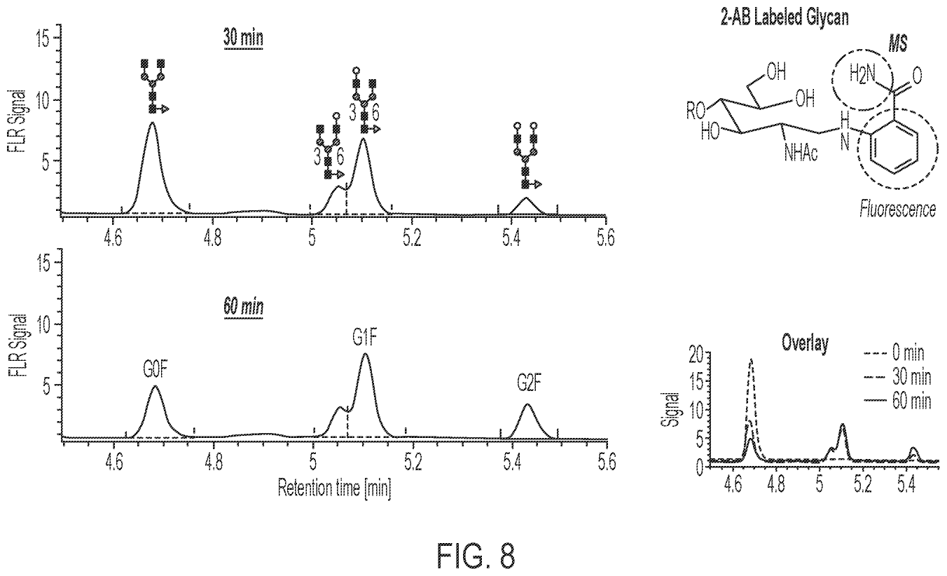

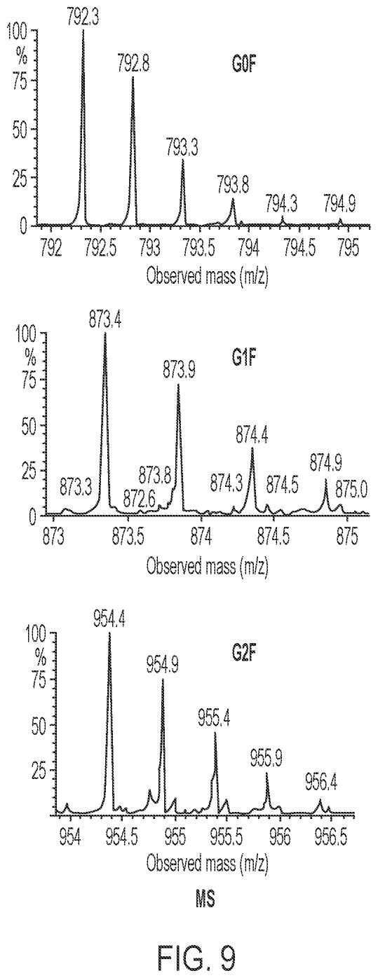

The quantitation of a 2-AB labelled glycan as a measure of β4GALT1 activity was first verified using 2-AB-G0F with the wildtype β4GALT1 enzyme. See FIG. 8. FIG. 8 displays a reduction in the signal for the 2-AB labeled G0F (the labeling substrate) and increase in the 2-AB labeled G1F and G2F (product) after 60 minutes compared to 30 minutes and 0 minutes, all captured using fluorescence detection. FIG. 9 displays the mass spectra obtained for G0F, G1F and G2F.

Further optimizations were carried out using the method of the present invention. A faster chromatography gradient was developed to reduce the length of the analysis while targeting the glycan of interest. Because the measurement is directed to the transfer of any galactose to the glycan, the separation of each isoform (with a galactose on one arm of the glycan, the other arm, or both) from each other is not necessary. Using this accelerated method, the longest retention time needed to be captured was reduced from 40 minutes (for the method of Example 2) to less than about 6 minutes.

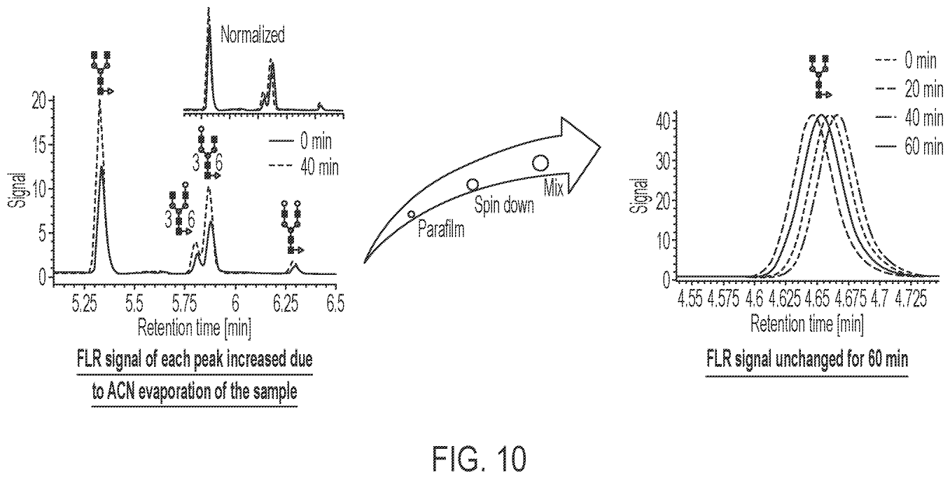

Due to the use of acetonitrile, sample evaporation was observed, which can interfere with peak assessment. This was fixed by sealing the samples with parafilm, spinning down and mixing them. See FIG. 10.

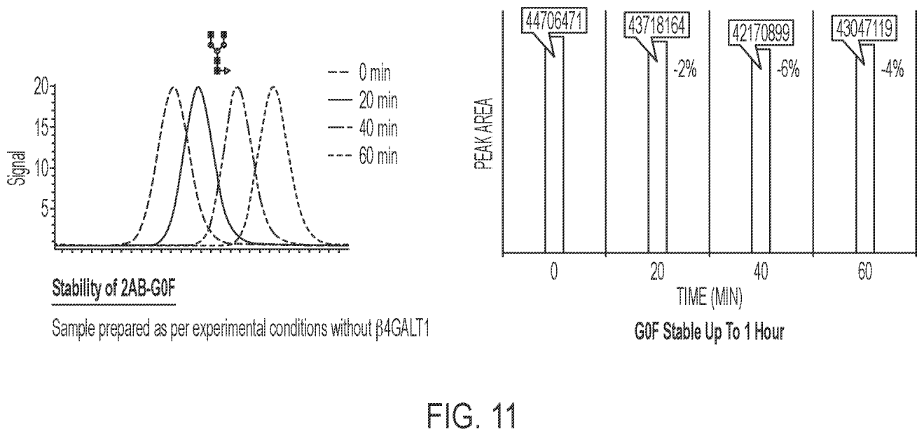

The stability of the labeling substrate was also assessed. FIG. 11 shows the fluorescence signal for 2-AB-G0F, prepared as per experimental conditions but without β4GALT1, at 0 minutes, 20 minutes, 40 minutes, and 60 minutes. It was observed that 2-AB-G0F was stable for at least about an hour.

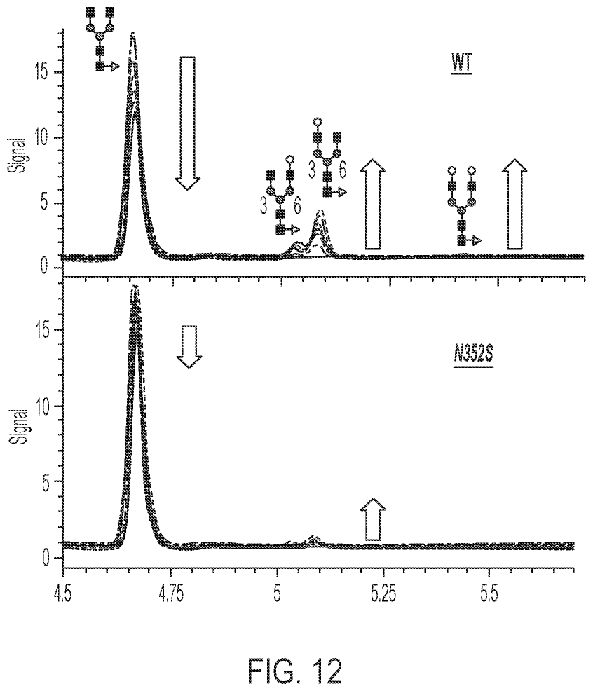

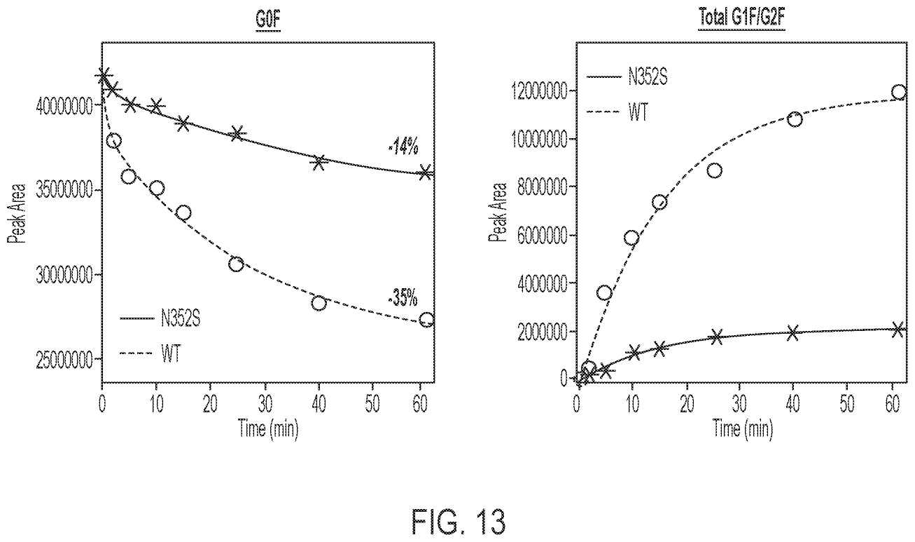

The difference in activity between the wildtype and mutant β4GALT1 was tested using the method of the present invention, and statistically assessed using a paired two-sided Student's t-test. The results are shown in FIG. 12 and FIG. 13. FIG. 12 and FIG. 13 show that the fluorescent signal for 2-AB-G0F declines substantially over time when incubated with wildtype β4GALT1 but less so with mutant β4GALT1, and conversely, the signal for the products 2-AB-G1F and 2-AB-G2F increase substantially over time when incubated with wildtype β4GALT1 but less so with mutant β4GALT1. The activity for the enzymes as determined by the method of the present invention is similar to the results seen in Montasser et al., demonstrating that the method of the present invention was capable of accurately quantitating glycosyltransferase activity.

As explained above, further advantages of the method of the present invention include that it can be carried out in less time than alternative methods; it allows for simultaneous measurement and direct comparison of the substrate (glycosyl acceptor) and the product; and, because it allows for quantitating free glycans and not a specific product or substrate, it can be generalized to measure the activity of any glycosyltransferase enzyme. The method is also not limited to 2-AB, but is amenable to other glycan labeling substrates.

What is claimed is:

1. A method to assay the activity of an enzyme, said method comprising:
contacting said enzyme with a donor substrate and a labeling substrate to form a sample;
incubating said sample for a predetermined amount of time;
contacting said incubated sample to a liquid chromatography column; and
quantifying an amount of said labeling substrate in said incubated sample after contacting said chromatography column to assay the activity of the enzyme.

2. The method of claim 1, further comprising contacting a mixture of said donor substrate and said labeling substrate to a liquid chromatography column and quantifying the amount of said labeling substrate in said incubated sample.

3. The method of claim 1, wherein the sample is incubated for two or more predetermined amounts of time, forming two or more samples, and the two or more incubated samples are contacted to a liquid chromatography column and amounts of said labeling substrate in said contacted samples are quantified.

4. The method of claim 1, wherein the enzyme is a mutated version of a naturally occurring enzyme.

5. The method of claim 1, wherein the enzyme is a naturally occurring enzyme.

6. The method of claim 1, wherein the enzyme is β4GALT1.

7. The method of claim 1, wherein enzyme catalyzes glycosylation.

8. The method of claim 1, wherein the enzyme catalyzes transfer of a sugar molecule onto a protein.

9. The method of claim 1, wherein the enzyme catalyzes transfer of a functional group onto a protein.

10. The method of claim 1, wherein the enzyme catalyzes glycosylation, the donor substrate provides a sugar molecule and said labeling substrate is capable of binding to the sugar molecule of the donor substrate.

11. The method of claim 1, wherein said labeling substrate is capable of being detected by use of mass spectrometry, fluorescence, chemiluminescence, bioluminescence, electrochemiluminescence, or radioactivity.

12. The method of claim 1, wherein said labeling substrate includes 2-aminobenzamide.

13. The method of claim 1, wherein said labeling substrate is quantified using mass spectrometry.

14. The method of claim 13, wherein said mass spectrometry is coupled online with said liquid chromatography column.

15. The method of claim 14, wherein said liquid chromatography column coupled with mass spectrometry is run under native conditions.

16. The method of claim 1, wherein the liquid chromatography column is an anion exchange column, a cation exchange column, a hydrophobic interaction chromatography column, a hydrophilic interaction chromatography column, a mixed mode chromatography column, or a protein A chromatography column.

17. The method of claim 1, wherein said enzyme is contacted with said donor substrate before contacting with said labeling substrate to form said sample.

18. The method of claim 1, wherein said enzyme is contacted with said labeling substrate before contacting with said donor substrate to form said sample.

19. A method to assay the activity of an enzyme, said method comprising:
contacting said enzyme with a donor substrate and a labeling substrate to form a sample, wherein said donor substrate and said labeling substrate forms a product;
incubating said product for a predetermined amount of time;
contacting said incubated product to a liquid chromatography column; and
quantifying an amount of said product present in said incubated sample after contacting said chromatography column to assay the activity of the enzyme.

20. The method of claim 19, further comprising contacting a mixture of said donor substrate and said labeling substrate liquid chromatography column and quantifying amount of said product in said incubated sample.

21. The method of claim 19, wherein the product is incubated for two or more predetermined amounts of time, forming two or more products, and the two or more incubated products are contacted to a liquid chromatography column and amounts of said product in said contacted products are quantified.

22. The method of claim 19, wherein the enzyme is a mutated version of a naturally occurring enzyme.

23. The method of claim 19, wherein the enzyme is a naturally occurring enzyme.

24. The method of claim 19, wherein the enzyme is β4GALT1.

25. The method of claim 19, wherein enzyme catalyzes glycosylation.

26. The method of claim 19, wherein the enzyme catalyzes transfer of a sugar molecule onto a protein.

27. The method of claim 19, wherein the enzyme catalyzes transfer of a functional group onto a protein.

28. The method of claim 19, wherein the enzyme catalyzes glycosylation, the donor substrate provides a sugar molecule and said labeling substrate is capable of binding to the sugar molecule of the donor substrate.

29. The method of claim 19, wherein said product is capable of being detected by use of mass spectrometry, fluorescence, chemiluminescence, bioluminescence, electrochemiluminescence, or radioactivity.

30. The method of claim 19, wherein said product includes 2-aminobenzamide.

31. The method of claim 19, wherein said product is quantified using mass spectrometry.

32. The method of claim 31, wherein said mass spectrometry is coupled online with said liquid chromatography column.

33. The method of claim 32, wherein said liquid chromatography column coupled with mass spectrometry is run under native conditions.

34. The method of claim 19, wherein the liquid chromatography column is an anion exchange column, a cation exchange column, a hydrophobic interaction chromatography column, a hydrophilic interaction chromatography column, a mixed mode chromatography column, or a protein A chromatography column.

35. The method of claim 19, wherein said enzyme is contacted with said donor substrate before contacting with said labeling substrate to form said product.

36. The method of claim 19, wherein said enzyme is contacted with said labeling substrate before contacting with said donor substrate to form said product.

37. A method for quantifying or characterizing the activity of a glycosyltransferase, comprising:
(a) incubating a mixture including a glycosyl donor, a glycosyl acceptor, and a glycosyltransferase, wherein said glycosyl acceptor includes a fluorescent label, and wherein said glycosyltransferase is capable of transferring a saccharide from said glycosyl donor to said glycosyl acceptor to form a product;
(b) subjecting said incubated mixture to chromatographic separation to separate said glycosyl acceptor from said product; and
(c) subjecting said separated glycosyl acceptor and said separated product to fluorescence spectroscopy to quantify or characterize the activity of said glycosyltransferase.

38. The method of claim 37, wherein said glycosyl donor is uridine diphosphate galactose.

39. The method of claim 37, wherein said glycosyl acceptor is a glycan.

40. The method of claim 37, wherein said glycosyl acceptor is a G0F glycan.

41. The method of claim 37, wherein said glycosyltransferase is Beta-1,4-galactosyltransferase 1.

42. The method of claim 37, wherein said fluorescent label is 2-aminobenzamide.

43. The method of claim 37, wherein said saccharide is galactose.

44. The method of claim 37, wherein said chromatographic separation is hydrophilic interaction chromatography separation.

45. The method of claim 37, further comprising subjecting said incubated mixture to ultraviolet detection.

46. The method of claim 37, further comprising subjecting said separated glycosyl acceptor and/or said separated product to mass spectrometry analysis.

47. The method of claim 37, wherein a duration of said incubating is from about 0 minutes to about 60 minutes, about 0 minutes, about 1 minute, about 2 minutes, about 5 minutes, about 10 minutes, about 15 minutes, about 25 minutes, about 30 minutes, about 40 minutes, about 50 minutes, or about 60 minutes.
